(19) United States
(12) Patent Application Publication
(10) Pub. No.: US 2026/0193304 A1
(43) Pub. Date: Jul. 9, 2026

(54) BIOMARKER FOR PREDICTING RISK OF DRUG ADDICTION RELAPSE AND ANIMAL MODEL WITH HIGH RELAPSE OF DRUG ADDICTION

(71) Applicant: KOREA INSTITUTE OF SCIENCE AND TECHNOLOGY, Seoul (KR)

(72) Inventors: Heh-In IM, Seoul (KR); Jin Hee BAE, Seoul (KR); Eun Mi HWANG, Seoul (KR)

(73) Assignee: KOREA INSTITUTE OF SCIENCE AND TECHNOLOGY, Seoul (KR)

(21) Appl. No.: 19/322,108

(22) Filed: Sep. 8, 2025

(30) Foreign Application Priority Data
Jan. 9, 2025 (KR) 10-2025-0003282

(51) Int. Cl. C07K 14/47; A01K 67/0276; A61K 49/00; C12N 15/113; C12N 15/85; C12N 15/86; G01N 33/50

(52) U.S. Cl. CPC C07K 14/4702; A01K 67/0276; A61K 49/0008; C12N 15/113; C12N 15/8509; C12N 15/86; G01N 33/5088; A01K 2217/058; A01K 2227/105; A01K 2267/03; C12N 2015/8527; C12N 2310/531; C12N 2750/14143

(57) ABSTRACT

The present disclosure has, for the first time, identified the relationship between the expression level of MeCP2 in D2R neurons of a ventral striatum and relapse of drug addiction, and provides this as a biomarker for predicting relapse of drug addiction. Furthermore, an animal model in which MeCP2 expression is suppressed specifically in D2R neurons of the ventral striatum has been developed, which is expected to greatly contribute to elucidating the mechanism of relapse of drug addiction and developing therapeutics therefor.

### CROSS-REFERENCE TO RELATED APPLICATION

This application claims the benefit of Korean Patent Application No. 10-2025-0003282, filed on Jan. 9, 2025, in the Korean Intellectual Property Office, the entire disclosure of which is incorporated herein by reference for all purposes.

### BACKGROUND

### 1. Field of the Invention

One or more embodiments relate to a biomarker for predicting the risk of relapse of drug addiction and an animal model with high relapse of narcotic addiction.

### 2. Description of the Related Art

Dopamine is synthesized by midbrain neurons located in the substantia nigra (SN) and the ventral tegmental area (VTA), and the synthesized dopamine is released and functions by binding to dopamine receptors present on the cell membrane to initiate intracellular signal transduction. Five subtypes of dopamine receptors have been identified and are categorized into two groups based on their structural and pharmacological properties: D1-like receptor group and D2-like receptor group. D1 and D5 receptors belong to the D1-like group, which is known to activate adenylate cyclase and promote the formation of intracellular cAMP. D2, D3, and D4 receptors belong to the D2-like group, which is known to inhibit cAMP formation.

Among these, the dopamine D2 receptor (D2R) plays a central role in various neurophysiological functions, including motor control, reward behavior, and the regulation of pituitary hormones, and is considered a key component of dopaminergic neurotransmission. In addition, the D2R can function as a presynaptic receptor among the dopamine receptors, and is highly expressed in dopaminergic neurons in the SN and VTA regions of the midbrain, serving as an autoreceptor. The D2R autoreceptor is known to be distributed in the somatodendritic region of dopaminergic neurons and to play a role in reducing neuronal excitability, or to be present at the terminals of dopaminergic neurons and to inhibit dopamine synthesis and release.

It is now a well-known fact that addictive drugs alter the mesolimbic mesocortical pathway in the dopamine circuit. In particular, repeated exposure to addictive drugs such as cocaine increases dopamine release in the nucleus accumbens (NAc) of the mesolimbic pathway, and these repeated stimuli induce synaptic changes in the mesolimbic system, which are presumed to be the neurobiological basis leading to addictive behavior to drugs. However, it can be stated that studies on actual synaptic changes in the mesolimbic system are still insufficient to draw a clear picture, despite many studies having been conducted and reported, due to the complexity of the phenomena and related factors.

In the past 2 to 3 years, research reports have begun to emerge that examine addictive behaviors and signals by activating or inhibiting medium spiny neurons (MSNs), which are cells throughout the striatum where dopamine receptors D1R/D2R are expressed, using optogenetics and genetic manipulation techniques, providing grounds to estimate the specific roles of each receptor.

In positron emission tomography (PET) imaging experiments conducted on humans, it has been revealed that D2R signaling is downregulated in specific brain regions (striatum) in the brains of actual drug addicts, especially cocaine addicts, suggesting that D2R is an important factor in regulating drug addiction. However, to understand the complex structural and functional synaptic changes that occur in addiction, it is necessary to develop and apply current techniques such as optogenetics and designer receptors exclusively activated by designer drugs (DREADDs) in a circuit-specific manner, and to adopt an integrated approach such as connectomics that combines molecular and cellular biology, fine optical imaging structure, and behavioral analysis. In addition, although studies focusing on related neurotransmitters that have been approached so far, especially receptors of dopamine or glutamate, are important, integrated research targeting the downstream signaling pathways of these receptors should also be conducted.

Drug addiction proceeds in a cyclical stage of intoxication→withdrawal→craving, and it is known that the relapse stage during each stage of drug addiction and the recovery stage of drug addiction is closely related to distinct epigenetic changes. The inventors of the present disclosure have diligently conducted research to elucidate the molecular neurological mechanisms related to relapse of drug addiction during the recovery stage of drug addiction and to meet the need to develop an animal model with high relapse of narcotic addiction for such research, thereby completing the present disclosure.

### SUMMARY

An aspect provides a level of MeCP2 in D2R cells of a ventral striatum as a biomarker for diagnosing individuals at high risk of relapse of drug addiction.

Another aspect provides an animal model with high relapse of drug addiction in which expression of MeCP2 is downregulated in D2R cells of a ventral striatum, a method of producing an animal model, and a drug screening method for preventing relapse of drug addiction, using the animal model.

However, technical goals to be achieved are not limited to those described above, and other goals not mentioned above are clearly understood by one of ordinary skill in the art from the following description.

To address the above goals, the present disclosure provides a biomarker for predicting the likelihood of relapse of drug addiction, including MeCP2 or an MeCP2 gene expressed in D2R neurons of a ventral striatum.

The biomarker exhibits a low expression level in individuals at high risk of relapse of drug addiction.

In addition, the present disclosure provides a pharmaceutical composition for preventing relapse of drug addiction, including as an active ingredient an agent capable of increasing an expression level of MeCP2 specifically in D2R neurons of a ventral striatum.

In an embodiment of the present disclosure, the MeCP2 gene may be DNA or RNA including a base sequence encoding MeCP2.

In another embodiment of the present disclosure, the vector may be a viral vector.

In another embodiment of the present disclosure, the viral vector may be one or more vectors selected from the group consisting of a retrovirus, a lentivirus, an adenovirus, an adeno-associated virus, and a hybrid vector.

The present disclosure also provides a method of producing an animal model with high relapse of drug addiction, including knocking down an MeCP2 gene specifically in D2R neurons in a ventral striatum of an individual.

In an embodiment of the present disclosure, the knocking down of the MeCP2 gene may be performed by injection of a viral vector expressing an RNAi sequence targeting the MeCP2 gene, and specifically, the RNAi may be shRNA.

In another embodiment of the present disclosure, the injection of the viral vector may be performed by stereotaxic surgery.

The present disclosure also provides an animal model with high relapse of drug addiction prepared by the above-described method. Here, the animal model is characterized in that expression of MeCP2 is downregulated in D2R cells of a ventral striatum.

The present disclosure also provides an animal model with high relapse of drug addiction in which expression of MeCP2 is suppressed specifically in D2R neurons in a ventral striatum.

The present disclosure also provides a drug screening method for preventing relapse of drug addiction, using the above-described animal model.

In an embodiment of the present disclosure, the drug screening method may include administering a candidate substance to the animal model, evaluating drug dependence after the candidate substance is administered, and selecting the candidate substance as a drug for preventing relapse of drug addiction when the drug dependence is significantly reduced compared to a control group.

In another embodiment of the present disclosure, the drug screening method may include administering a candidate substance to the animal model, measuring an expression level of MeCP2 in D2R neurons of a ventral striatum sample from the animal model after the candidate substance is administered, and selecting the candidate substance as a drug for preventing relapse of drug addiction when there is no significant difference in the expression level of MeCP2 compared to a control group.

In another embodiment of the present disclosure, the measuring of the expression level of MeCP2 may be performed by a method selected from the group consisting of reverse transcription polymerase chain reaction, competitive reverse transcription polymerase chain reaction, real-time reverse transcription polymerase chain reaction, RNase protection assay, Northern blotting, DNA chip, protein chip analysis, immunoassay, ligand binding assay, matrix-assisted laser desorption/ionization time-of-flight (MALDI-TOF) mass spectrometry, surface-enhanced laser desorption/ionization time-of-flight (SELDI-TOF) mass spectrometry, radioimmunoassay, radial immunodiffusion, Ouchterlony immunodiffusion, rocket immunoelectrophoresis, immunohistochemistry, complement fixation assay, two-dimensional electrophoresis, liquid chromatography-mass spectrometry (LC-MS), liquid chromatography-mass spectrometry/mass spectrometry (LC-MS/MS), Western blot, enzyme-linked immunosorbent assay (ELISA), and sandwich ELISA.

In an embodiment of the present disclosure, the drug may be a narcotic, and specifically may be cocaine.

Additional aspects of embodiments will be set forth in part in the description which follows and, in part, will be apparent from the description, or may be learned by practice of the disclosure.

The present disclosure has, for the first time, identified the relationship between the expression level of MeCP2 in D2R neurons of a ventral striatum and relapse of drug addiction, thereby providing the expression level of MeCP2 in the neurons as a biological indicator for predicting the risk of relapse of drug addiction, and providing an animal in which MeCP2 expression is downregulated in the neurons as an animal model with high relapse of drug addiction, which is expected to greatly contribute to elucidating the mechanism of relapse of narcotic addiction and developing therapeutics therefor.

### BRIEF DESCRIPTION OF THE DRAWINGS

These and/or other aspects, features, and advantages of the invention will become apparent and more readily appreciated from the following description of embodiments, taken in conjunction with the accompanying drawings of which:

FIG. 1A is a schematic diagram of Cre-loxP system used to downregulate MeCP2 expression in D2R cells.

FIG. 1B is a schematic diagram of stereotaxic surgery in which an MeCP2 shRNA-expressing viral vector is administered to downregulate MeCP2 expression in D2R cells of a ventral striatum.

FIG. 2A is a schematic diagram of a conditioned place preference (CPP) experiment conducted after the stereotaxic surgery.

FIG. 2B is a diagram of the CPP experiment.

FIG. 2C shows a diagram and photograph of the CPP experimental apparatus.

FIG. 2D shows the results of the CPP experiment. a: A graph comparing changes in drug-associated CPP across experimental stages between the control group (CTR) and the shMeCP2 group. b: A bar graph comparing drug preference between the two groups after drug conditioning (post-test) and after relapse induction (reinstatement). c: A heatmap showing the positions occupied by mice in the CTR and the shMeCP2 group during the reinstatement stage. d: A result demonstrating that there was no difference in CPP between the two groups prior to drug conditioning. e: A result indicating that drug preference similarly decreased in both groups on day 5 of extinction learning.

FIG. 3 shows the results of open field test (OFT), novel object recognition (NOR) test, and elevated plus maze (EPM) test in an animal model with high relapse of drug addiction. a: A result from the NOR test indicating that there is no significant difference in cognitive function between the two groups. b: A result from the OFT measuring the duration spent in the center zone, indicating that anxiety levels are comparable between the two groups. c: A result from the OFT measuring the total distance moved, demonstrating that overall locomotor activity is similar between the two groups. d: A result from the EPM test measuring the percentage of entries into the open arms, indicating that there is no difference in anxiety levels between the two groups.

### DETAILED DESCRIPTION

To date, no research results have been presented that demonstrate a relationship between relapse of narcotic addiction and regulation of the MeCP2 gene.

The inventors of the present disclosure prepared genetically modified mice in which MeCP2 expression was downregulated specifically in D2R neurons by introducing MeCP2 shRNA into the ventral striatum of D2R-Cre mice using a Cre-loxP system. A conditioned place preference (CPP) test was conducted, and although no significant differences in preference were observed during the pretest, posttest, and extinction phases, the genetically modified mice exhibited a higher level of drug dependence compared to the control group when preference was assessed the day after administration of a priming dose following extinction learning.

Based on this, the inventors confirmed that low expression of MeCP2 in D2R neurons of a ventral striatum may lead to relapse of drug addiction due to drug dependence in individuals undergoing treatment for drug addiction. Therefore, the present disclosure provides an indicator of MeCP2 expression level in D2R neurons of a ventral striatum as a biomarker for diagnosing individuals at high risk of relapse of drug addiction.

In addition, the present disclosure provides a method of providing information for diagnosing individuals at high risk of relapse of drug addiction and a method of diagnosing individuals at high risk of relapse of drug addiction, including measuring of the MeCP2 expression level in D2R neurons of a ventral striatum in individuals in the rehabilitation stage of drug addiction.

More specifically, the method of providing information for diagnosing individuals at high risk of relapse of drug addiction and the method of diagnosing individuals at high risk of relapse of drug addiction according to an embodiment of the present disclosure may include: acquiring a biological sample derived from the ventral striatum of a subject; measuring an expression level of MeCP2 in D2R cells in the sample; comparing the MeCP2 expression level in the sample of the subject with that in a sample derived from an individual who has successfully completed rehabilitation from drug addiction; and determining that the subject is an individual with a high possibility of relapse of narcotic addiction if the MeCP2 expression level in the subject's sample is lower than that in the sample from the individual who has successfully completed rehabilitation from drug addiction.

The measuring of the expression level of MeCP2 may be performed by a method selected from the group consisting of reverse transcription polymerase chain reaction, competitive reverse transcription polymerase chain reaction, real-time reverse transcription polymerase chain reaction, RNase protection assay, Northern blotting, DNA chip, protein chip analysis, immunoassay, ligand binding assay, matrix-assisted laser desorption/ionization time-of-flight (MALDI-TOF) mass spectrometry, surface-enhanced laser desorption/ionization time-of-flight (SELDI-TOF) mass spectrometry, radioimmunoassay, radial immunodiffusion, Ouchterlony immunodiffusion, rocket immunoelectrophoresis, immunohistochemistry, complement fixation assay, two-dimensional electrophoresis, liquid chromatography-mass spectrometry (LC-MS), liquid chromatography-mass spectrometry/mass spectrometry (LC-MS/MS), Western blot, enzyme-linked immunosorbent assay (ELISA), and sandwich ELISA, but is not limited thereto.

In addition, the present disclosure provides a method of producing an animal model with high relapse of drug addiction, including downregulating expression of an MeCP2 gene in D2R neuronal cells of a ventral striatum of an individual.

The method of producing an animal model may include: preparing a viral vector expressing an RNAi sequence targeting the MeCP2 gene; and knocking down the MeCP2 gene by injecting the vector into the ventral striatum of an individual.

In the present disclosure, the term “vector” refers to a DNA construct that contains a DNA sequence operably linked to suitable regulatory sequences enabling the expression of the DNA in a host, without limitation. The vector may be a viral vector. Non-limiting examples of the viral vector may include retrovirus, lentivirus, adenovirus, adeno-associated virus (AAV), and hybrid vector, and preferably, the viral vector may be an AAV. The AAV can efficiently infect habenular neurons regardless of cell division and has minimal cytotoxicity and pathogenicity in humans or animals, making it effective for producing the animal model of the present disclosure.

Additionally, in the present disclosure, the method of injecting the viral vector into the ventral striatum of an individual is not particularly limited, but it is preferably performed using stereotaxic surgery to minimize brain damage and enable precise viral injection.

In addition, the present disclosure may provide a non-human animal model with high relapse of drug addiction, in which expression of MeCP2 is reduced specifically in D2R neurons of a ventral striatum.

The animal model is not limited as long as the animal model is produced by a method capable of reducing the expression of MeCP2 specifically in D2R neurons of a ventral striatum.

In the present disclosure, RNAi that interferes with the expression of MeCP2 in D2R neurons of a ventral striatum of an individual is not limited as long as the RNAi includes a sequence complementary to all or part of the MeCP2 gene and interferes with its activity or expression.

Furthermore, the present disclosure may also provide a drug screening method for preventing relapse of drug addiction, using the above-described animal model.

The drug screening method may include administering a candidate substance to the animal model, evaluating narcotic dependence after the candidate substance is administered, and selecting the candidate substance as a drug for preventing relapse of narcotic addiction when the drug dependence is significantly reduced compared to a control group.

In the evaluating of the narcotic dependence, the drug dependence may be evaluated through the CPP test, but is not limited thereto.

Additionally, the drug screening method may include administering a candidate substance to the animal model, measuring an expression level of MeCP2 in D2R neurons of a ventral striatum sample from the animal model after the candidate substance is administered, and selecting the candidate substance as a drug for preventing relapse of narcotic addiction when there is no significant difference in the expression level of MeCP2 compared to a control group.

In the drug screening method, the control group refers to animals in which MeCP2 expression in D2R neurons of a ventral striatum is not regulated.

In the present disclosure, the subject of diagnosis concerns the possibility of relapse of drug addiction, and refers to identifying individuals exhibiting high drug dependence after drug withdrawal in drug-addicted individuals. In the present disclosure, such individuals are classified as individuals at high risk of relapse of drug addiction.

Additionally, the animal model of the present disclosure, which exhibits high drug dependence after drug administration and subsequent withdrawal, is referred to as an animal model with high relapse of drug addiction.

In addition, in the present disclosure, the term “individual” refers to a mammal including humans. However, in the animal model provided for research on the mechanism of relapse of drug addiction and for screening drugs for its prevention and treatment, the individual refers to a non-human mammal, preferably a rodent, and more preferably Mus musculus (mouse) or Rattus norvegicus (rat). Mice and rats are advantageous as laboratory animals due to their ease of breeding, rapid reproduction, and low cost. In particular, mice are inherently highly active, making them more suitable for evaluating drug dependence through behavioral experiments.

The present disclosure can be subjected to various modifications and can have various embodiments, and thus, specific embodiments are illustrated in the drawings and described in detail in the following detailed description. However, this is not intended to limit the present disclosure to specific embodiments, and it should be understood to include all modifications, equivalents, or substitutes that fall within the idea and scope of the present disclosure. In the description of embodiments, detailed description of well-known related technologies will be omitted when it is deemed that such description will cause ambiguous interpretation of the present disclosure.

### EXAMPLES

### Example 1. Production of Animal Model with High Relapse of Narcotic Addiction

Genetically modified mice expressing MeCP2 shRNA were produced by using a Cre-loxP system, in which MeCP2 expression was reduced specifically in D2R neurons in the ventral striatum of D2R-Cre mice.

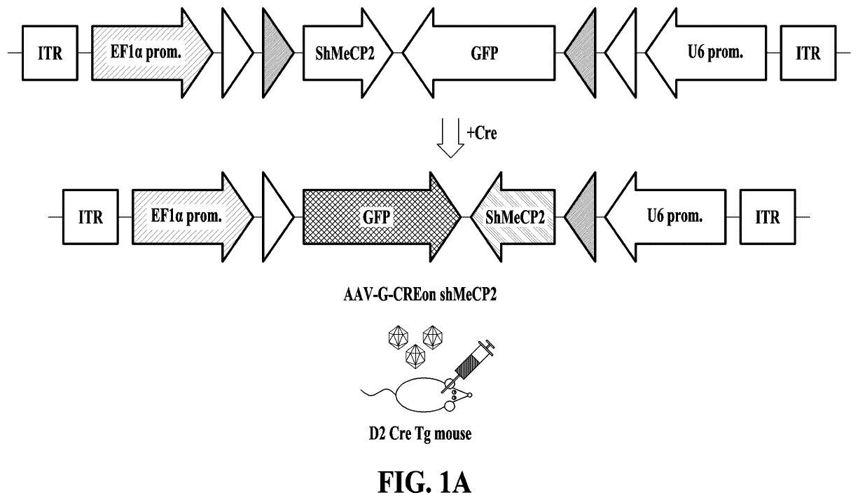

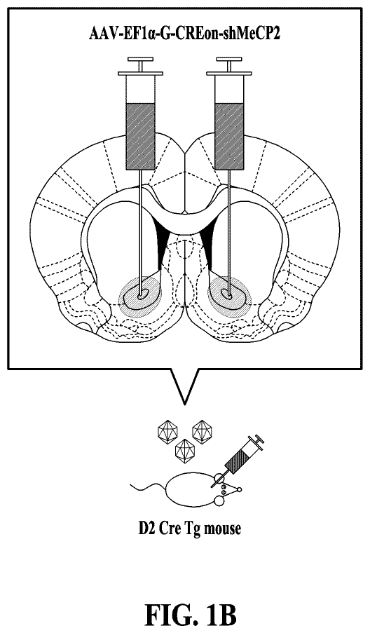

Specifically, an AAV containing a double floxed inverted open reading frame (DIO) of mouse MeCP2 shRNA or eGFP sequence was administered to genetically modified mice that express Cre recombinase specifically in D2R neurons (B6.FVB(Cg)-Tg(Drd2-cre)ER44Gsat/Mmucd, RRID: MMRRC_032108-UCD) (FIG. 1A). AAV(DJ/8)-G-CREon-shMeCP2 or AAV(DJ/8)-G-CREon-scrambled shRNA virus was injected into the mice so that Cre recombinase would be expressed specifically in D2R neurons (FIG. 1B).

Two weeks after stereotaxic surgery, a cognitive behavioral test was conducted to confirm whether the genetically modified mice exhibited symptoms of relapse of narcotic addiction.

### Example 2. Validation of Animal Model With High Relapse of Narcotic Addiction

### 2-1. Cognitive Behavioral Test for Evaluation of Relapse of Narcotic Addiction

### 2-1-1. CPP

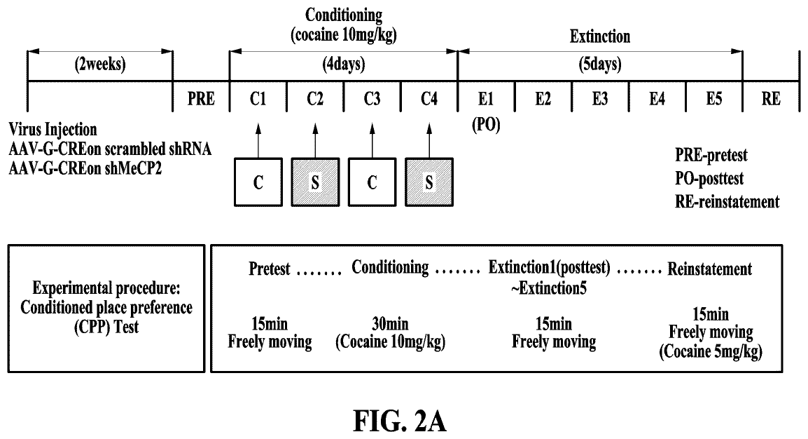

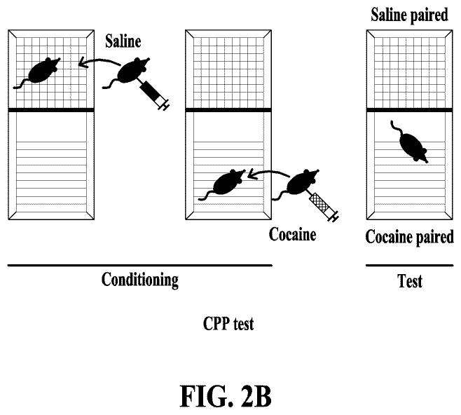

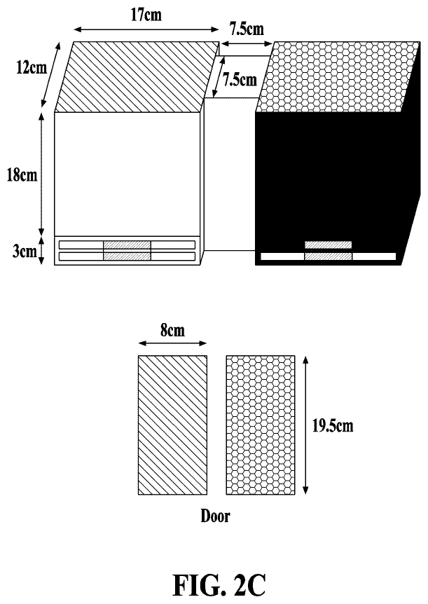

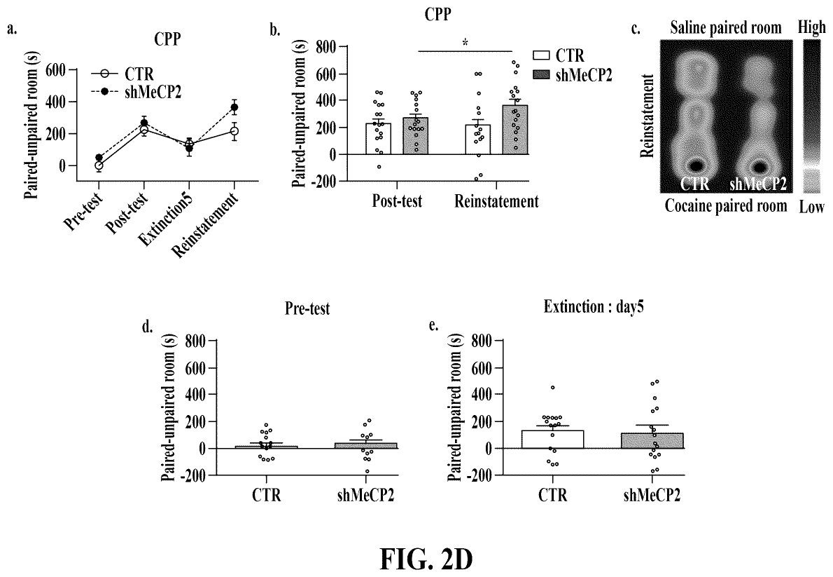

Two weeks after surgery, a CPP experiment was performed (FIG. 2A). The CPP chamber was designed and constructed as shown in FIG. 2C. The center room was designed to be smaller in size so that the mice would stay longer on average in the two side rooms among the three rooms. To prevent differences in time spent between the two side rooms, i.e., to avoid bias toward one particular room, the wall or floor colors, patterns, and brightness were designed in a balanced manner. After allowing free movement among the three rooms for 15 minutes, the preference for one side room was calculated using the formula: [time spent in room A] (randomly selected room)/[time spent in rooms A +B]×100, and mice showing a preference exceeding 65% as an integer value for the corresponding room were judged to have a high preference for that room and were excluded from the experiment.

The CPP experiment is a behavioral experiment in the format of a classical Pavlovian conditioning model. In this experiment, a specific space or situation is associated with the administration of an addictive drug, while another specific space or situation is associated with the administration of a control substance such as saline. After this conditioning training, the animal's preference for a specific situation or space is analyzed in a test performed without drug administration. The degree of preference for the place or situation associated with the addictive drug represents the Pavlovian reinforcement effect of the drug, and is ultimately used to measure drug craving.

Specifically, the experiment was conducted by first performing a pretest (PRE) phase two weeks after surgery, in which all doors of the CPP chamber were opened and the mice were allowed to move freely among the three rooms to investigate preference for a specific room, and then performing a conditioning phase in which the effect of a drug (cocaine) or a non-drug (saline) injection was associated with a specific room. On the first day of the conditioning phase, 10 mg/kg of cocaine was injected into the mice and the mice were confined in a specific room (excluding the center room) for 30 minutes. On the second day, saline was injected, and the mice were confined in the other room for 30 minutes. This procedure was repeated over four days, with each condition performed twice. On the day following the conditioning, all three doors were opened, and the mice were allowed to move freely to evaluate the degree of drug preference (dependence) (posttest, PO). The PO process was repeated for five days to perform an extinction (EX) phase, in which the mice learned that no reward was provided in the previously drug-associated place. Following the EX phase, on the next day, a priming dose (5 mg/kg) of the drug was administered, and the mice were allowed to move freely among the three rooms, and the degree of relapse was measured.

### 2-1-2. Open Field Test (OFT)

The OFT is a method of measuring locomotor activity and anxiety level by observing exploratory behavior when mice are exposed to a new space the day after being subjected to restraint stress. After the restraint stress, the mice were allowed to move freely within the box for 30 minutes to observe locomotor activity, and the time the mice spent in the center area of the open field was measured to assess the anxiety level.

### 2-1-3. Novel Object Recognition Test (NOR)

Mice generally show a tendency to exhibit interest in novel objects. After allowing the mice to experience two identical objects (old objects) in a specific space, one of the two objects was replaced with a new object the next day, and the degree of the mice's interest (sniffing) in the new object was checked. A high level of interest in the new object indicates that the memory of the previous object is well maintained. Conversely, a relatively low level of interest in the new object indicates that memory impairment due to stress has occurred in the mice.

### 2-1-4. Elevated Plus Maze (EPM)

The anxiety level was measured by recording the time the mice stayed in the open arms among the two open white arms and two closed black arms of the maze.

### 2-2. Experimental Results

According to the results of the CPP test, no difference was observed between the two groups in the PO and EX phases (a, d, and e in FIG. 2D). However, in the animal model with high relapse reduced MeCP2 in D2R neurons of the ventral striatum, a higher relapse rate was observed compared to the control group. This suggests that the possibility of reestablishing drug dependence after partial extinction of the reinforcement effect of the drug is significantly increased (a to c in FIG. 2D).

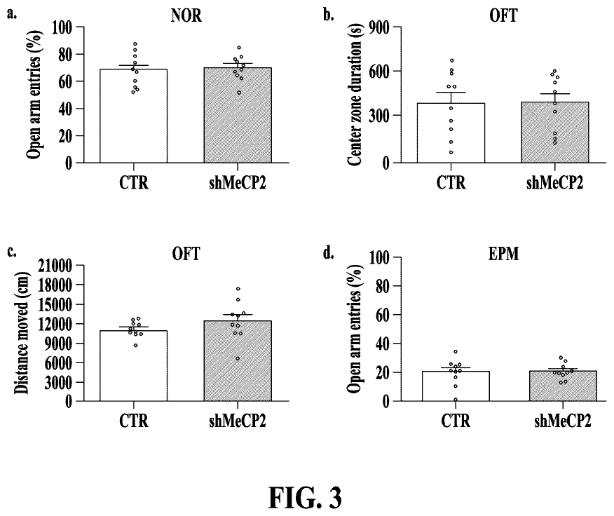

Meanwhile, the animal model with high relapse of narcotic addiction showed no significant difference from the control group in locomotor activity and cognitive function (FIG. 3).

While the embodiments have been described with reference to the limited drawings, it will be apparent to one of ordinary skill in the art that various alterations and modifications can be made from the above description. For example, suitable results may be achieved if the described techniques are performed in a different order, and/or if components in a described system, structure, device, or circuit are combined in a different manner, and/or replaced or supplemented by other components or their equivalents.

Therefore, other implementations, other embodiments, and equivalents to the claims are also within the scope of the following claims.

The present disclosure was completed with the support of the following project: Project Unique Number RS-2024-00332024 by the Korea Institute of Science and Technology.

What is claimed is:

1. A pharmaceutical composition for preventing relapse of drug addiction, comprising as an active ingredient an agent capable of increasing an expression level of MeCP2 specifically in D2R neurons of a ventral striatum,
wherein the agent is a vector comprising an MeCP2 gene.

2. The pharmaceutical composition of claim 1, wherein the MeCP2 gene is DNA or RNA comprising a base sequence encoding MeCP2.

3. The pharmaceutical composition of claim 1,
wherein the vector is a viral vector, and
the viral vector is one or more vectors selected from the group consisting of a retrovirus, a lentivirus, an adenovirus, an adeno-associated virus, and a hybrid vector.

4. A method of producing an animal model with high relapse of drug addiction, the method comprising knocking down an MeCP2 gene specifically in D2R neurons in a ventral striatum of a non-human subject.

5. The method of claim 4, wherein the knocking down of the MeCP2 gene is performed by injection of a viral vector expressing an RNAi sequence targeting the MeCP2 gene.

6. The method of claim 5, wherein the injection of the viral vector is performed by stereotaxic surgery.

7. An animal model with high relapse of drug addiction in which expression of MeCP2 is suppressed specifically in D2R neurons in a ventral striatum.

8. A drug screening method for preventing relapse of drug addiction, using the animal model of claim 7.

9. The drug screening method of claim 8, comprising:
administering a candidate substance to the animal model;
evaluating drug dependence after the candidate substance is administered; and
selecting the candidate substance as a drug for preventing relapse of drug addiction when the drug dependence is significantly reduced compared to a control group.

10. The drug screening method of claim 9, wherein the evaluating of the drug dependence is achieved by performing a conditioned place preference (CPP) experiment.

11. The drug screening method of claim 8, comprising:
administering a candidate substance to the animal model;
measuring an expression level of MeCP2 in D2R neurons of a ventral striatum sample from the animal model after the candidate substance is administered; and
selecting the candidate substance as a drug for preventing relapse of drug addiction when there is no significant difference in the expression level of MeCP2 compared to a control group.

12. The drug screening method of claim 11, wherein the measuring of the expression level of MeCP2 is performed by a method selected from the group consisting of reverse transcription polymerase chain reaction, competitive reverse transcription polymerase chain reaction, real-time reverse transcription polymerase chain reaction, RNase protection assay, Northern blotting, DNA chip, protein chip analysis, immunoassay, ligand binding assay, matrix-assisted laser desorption/ionization time-of-flight (MALDI-TOF) mass spectrometry, surface-enhanced laser desorption/ionization time-of-flight (SELDI-TOF) mass spectrometry, radioimmunoassay, radial immunodiffusion, Ouchterlony immunodiffusion, rocket immunoelectrophoresis, immunohistochemistry, complement fixation assay, two-dimensional electrophoresis, liquid chromatography-mass spectrometry (LC-MS), liquid chromatography-mass spectrometry/mass spectrometry (LC-MS/MS), Western blot, enzyme-linked immunosorbent assay (ELISA), and sandwich ELISA.
